(19) United States
(12) Patent Application Publication
(10) Pub. No.: US 2019/0133947 A1
(43) Pub. Date: May 9, 2019

(54) Pharmaceutical Peptides and Rhamnolipid Liposomes

(71) Applicant: Keith DeSanto, St Petersburg, FL (US)

(72) Inventor: Keith DeSanto, St Petersburg, FL (US)

(21) Appl. No.: 15/984,331

(22) Filed: May 19, 2018

Related U.S. Application Data
(60) Provisional application No. 62/517,264, filed on Jun. 9, 2017.

(51) Int. Cl. A61K 9/127; A61K 38/16; A61K 31/7028

(52) U.S. Cl. CPC A61K 9/1272; A61K 31/7028; A61K 38/164

(57) ABSTRACT

This invention is about internalization of a peptide inside a rhamnolipid liposome. In this invention, the chemically synthesized peptide was ParE3, an analogue from ParE protein that acts on a Toxin-Antitoxin system. This peptide is able to inhibit DNA Gyrase and Topoisomerase IV (Topo IV) activities, blocking the DNA bacterial replication, regulating its cell growth for new antimicrobial drugs.

Rhamnolipids are one of the most important biosurfactant representers and are mainly produced by the fermentation rote of Pseudomonas aeruginosa, but they also can be produced by Rhodotorula taiwanensis, Lactobacillus plantarum, Pseudomonas rhizopila, Pseudomonas chlororaphis and Burkholderia sp. They are recognized as a “green production” due to their low environmental cytotoxicity, but they also have high emulsification potential and antimicrobial activities. By their amphipacity they can be used as liposomes.

### Rhamnolipids Production

The production medium consisted of a Ca-free mineral salt solution with 15.0 g/L NaNO₃, 0.5 g/L MgSO₄×7 H2O, 1.0 g/L KCl and as a phosphate source 0.3 g/L K₂HPO₄. As sole carbon source soybean oil with a starting concentration of 250 g/L was used and 1 mL/L of the above mentioned trace element solution was added.

The trace element solution contained 2.0 gIL sodium citratex2 H₂O, 0.28 g/L FeCl₃×6 H₂O, 1.4 g/L ZnSO₄×7 H₂O, 1.2 g/L CoCl₂×6 H₂O, 1.2 g/L CuSO₄×5 H2O, and 0.8 g/L MnSO₄×6 H₂O.

The fermentation was carried out at 37° C., pH 6.9, and the process was carried out for 158 h.

The rhamnolipid produced was purified by acidification and then a extraction was carried out using ethyl acetate.

The molecular weight of the rhamnolipid is between 475 g/mol and 677 g/mol.

Rhamnolipid Liposome Production

Vesicles were prepared in a PBS solution (pH 7.2-7.4) at a rhamnolipid, cholesterol and phosphatidylcholine final concentrations determined by Table 1. Firstly, each lipid were solubilized in chloroform, the solvent was evaporated by N2, and in a vacuum bomb for 18 hours, to eliminate any chloroform residues. Then, the obtained films were hydrated with PBS solution (pH 7.2-7.4), the samples were vortexed and sonicated for 6 minutes by 21% of amplitude or extruded 30 times in a 0.1 pm membrane.

TABLE 1
Composition of the vesicles

| | Rhamnolipid | | |
|---|---|---|---|
| Formulation | (mmol · L^{I}) | Cholesterol (mmol · L^{I}) | PC* (mmol · L⁻¹) |
| A | 2.6 | | |
| B | 2.6 | | 0.3 |
| C | 2.6 | 0.1 | |
| D | 2.6 | 0.1 | 0.3 |

PC*: phosphatidylcholine

### Synthesis, Purification and Identification of Peptides

ParELC3 was synthesized by Solid Phase Fmoc strategy, using a Rink-Amide MBLIA resin and activated by DIC and HOBt. Then, it was acetylated with anidride acetic. The cleavage was done with TFA/water/EDTAhioanisole (94:2.5:2.5:1) and ether. After it, ParELC3 was purified by HPLC (reverse phase) using a C18 column. Finally, the peptide was identified by mass spectrometry (ESI-MS Ion trap). To all experiments we used 100 μM of ParELC3

### Physical and Chemical Measures of Liposomes

Dynamic light scattering (DLS) was used to measure the particle size and polydispersity of liposomes composed by formulations A, B, C and D. The DLS (Zetasizer—Malwern) was used at 173°, at controlled temperature (25±1° C.). Electrophoretic mobility of liposomes was measured by Zeta Potential, using the dynamic light scattering (Zetasizer—Malvern).

The morphology and organization of liposomes were evaluated by TEM. For this study, samples were placed on a cooper grid and observed by using the staining-negative technique, where a drop of 1% (w/v) aqueous solution of uranyl acetate was added. The samples were imaged under a transmission electron microscope (IEOL JEM-100CX2) with an acceleration of 100 kv. The diameter of the liposomes was then determined by ImageJ software.

### Efficiency of Encapsulation (EE %)

The efficiency of encapsulation (EE %) study was evaluate by AMICON (50 kDa) centrifugation at 14.000×g during 14 minutes. Non-encapsulate peptide was able to cross the membrane and the solution was monitored by UV-Vis (280 nm). The concentration of peptide was done by a Lambert-Beer curve and efficiency of encapsulation was calculated by

X=(Non-encapsulate Concentration Peptide×100)/(Initial Concentration of Peptide)

### Microbiological Assays:

To determine the growth cell inhibition of Escherichia coli 0157:H17 (ATCC 43895) and Staphyllococos aureus (ATCC 14458) by rhamnolipids liposomes entrapped with ParELC3 a National Committee for Clinical Laboratory Standards (CLSI, 2006) microdilution method was used.

1. Using a peptide inside a Rhamnolipid Liposome for a pharmaceutical application.

2. Using claim 1, the chemically synthesized peptide is ParELC3, an analogue from ParE protein that acts on a Toxin-Antitoxin system.

3. Using claim 1 to inhibit DNA Gyrase and Topoisomerase IV (Topo IV) activities, blocking the DNA bacterial replication and regulating its cell growth for new antimicrobial applications.

4. Using claim 1, the antimicrobial application is impaired by its difficult bacterial cell membrane permeability.

5. Using claim 1, the antimicrobial application creates cell membrane permeability.

6. Using a peptide inside a rhamnolipid liposome for various applications.

7. Using claim 6, the chemically synthesized peptide is ParELC3, an analogue from ParE protein that acts on a Toxin-Antitoxin system.

8. Using claim 6, to inhibit DNA Gyrase and Topoisomerase IV (Topo IV) activities, blocking the DNA bacterial replication and regulating its cell growth for new antimicrobial applications.

9. Using claim 6, the antimicrobial application is impaired by its difficult bacterial cell membrane permeability.

10. Using claim 6, the antimicrobial application creates cell membrane permeability.

11. Using a biosurfactant and peptide analogue from ParE and antimicrobial application.

12. ParELC3 peptide internalization into rhamnolipid liposomes increases cell permeability and bioavailability.

13. Using claim 12, microbial inhibition is obtained.

14. Using claim 1 where the application is to treat wounds and burns in humans and animals

15. Using claim 1 where the application is to treat diseases affecting humans and animals.
